(19) United States
(12) Patent Application Publication
(10) Pub. No.: US 2005/0061335 A1
(43) Pub. Date: Mar. 24, 2005

(54) Irrigation solution for use in ultrasound energy assisted surgery

(76) Inventor: Morris Topaz, Kibutz Kamat Hacovesh (IL)

Correspondence Address: FOLEY AND LARDNER;SUITE 500, 3000 K STREET NW, WASHINGTON, DC, 20007, US

(21) Appl. No.: 10/840,657

(22) Filed: May 7, 2004

Related U.S. Application Data
(63) Continuation-in-part of application No. 10/686,730, filed on Oct. 17, 2003.
Application No. 10/686,730 is a division of application No. 09/478,363, filed on Jan. 6, 2000, now abandoned.

(30) Foreign Application Priority Data
Jan. 7, 1999 (IL) 127.967

(51) Int. Cl. A61B 19/00

(52) U.S. Cl. 128/898

(57) ABSTRACT

The invention provides a biocompatible, injectable sterile aqueous solution for use in high-intensity-ultrasound-energy-assisted surgery comprising antioxidants selected from the group consisting of ascorbic acid, glutathione and mixtures thereof in a buffered biocompatible solution for the reduction and limitation of reactive oxygen species.

### FIELD OF INVENTION

The present invention relates to a biocompatible, injectable sterile aqueous solution as well as to an irrigating or wetting solution for use in high-intensity-ultrasound-energy-assisted surgery. More specifically the irrigation solution of the present invention contains a water soluble antioxidant selected from the group consisting of ascorbic acid (AA), gluthatione and other water soluble antioxidants and mixtures thereof in a buffered, physiological pH solution, for the reduction and limitation of the concentration of radicals and sonochemistry reactions.

The present specification is a continuation in part of U.S. Ser. No. 10/686,730 filed Oct. 17, 2003 which in turn is a division of U.S. Ser. No. 09/478,363 filed Jan. 6, 2000 and now abandoned.

In U.S. Ser. No. 10/686,730 there is described and claimed a method for using a biocompatible, injectable aqueous solution in high intensity ultrasound energy assisted surgery at greater than 10 W/cm²CW at 20-100 kHz with an amplitude of 60-320%, wherein said solution comprises a gas selected from the group consisting of carbon dioxide, nitrogen, or mixtures thereof.

Said specification also discloses that said solution can comprise a water soluble scavenger which scavenger can be vitamin C otherwise known as ascorbic acid. It has now been discovered according to the present invention that ascorbic acid by itself and other similar antioxidants even without the presence of a gas selected from the group consisting of carbon dioxide, nitrogen or mixtures thereof is effective in reducing corneal endothelial cell loss secondary to high-energy ultrasound energy during phacoemulsification surgery by providing antioxidant protection against corneal damage by free radicals.

More specifically however according to the present invention it has now been discovered that the addition of antloxidents for this purpose results in the lowering of the pH of the irrigating solution to a harmful acidic level causing damage to the corneal endothelium through direct chemical change and that this damage can be avoided without obviating the advantageous affects of the antioxidants by adding a buffer to bring the solution to an acceptable pH range close to neutral.

Thus according to the present invention there is now provided a biocompatible, injectable aqueous solution for use in high-intensity-ultrasound-energy-assisted surgery comprising antioxidants selected from the group consisting of ascorbic acid, glutathione and mixtures thereof in a buffered biocompatible solution for the reduction and limitation of reactive oxygen species.

In an especially preferred embodiment of the present invention there is now provided a biocompatible, injectable aqueous irrigation solution for use in phacoemulsification comprising ascorbic acid in a buffered biocompatible solution for the reduction and limitation of reactive oxygen species

### BACKGROUND OF THE INVENTION

Ultrasound energy has been extensively used in the medical field, mainly for diagnostic and therapeutic purposes It has been utilized for diagnosis in medicine mainly at a sonar pulse mode of very short duration and of high frequency and peak intensity, or at low intensities of 10⁻² to 10⁻³ W/cm² CW, and at a high frequency In Doppler mode. Ultrasound is used in physical therapy in intensities of up to 3-5 W/cm² CW, for a short exposure time. High-intensity ultrasound energy (HIUE) is applied in ablative surgery instrumentation, such as those used in phacoemulsification in ophthalmology, recanalization and thrombus dissolution in peripheral arteries, in neurosurgery, hepatic, renal, prostate and bladder resections, as well as in extracorporeal shock wave lithotripter, utilize higher power intensities that are above 10 W/cm²CW. The long-term safety of diagnostic ultrasonic equipment in clinical use was questioned and evaluated with no significant effects demonstrated at intensities below 100 mW/cm² spatial and temporal peak average intensity.

Phacoemulsification utilizes HIUE to fragment and emulsify the cataractous lens. In order to avoid the thermal effect resulting from the exposure of soft tissue to HIUE, a physiological, balanced salt solution (BSS), (irrigating/wetting solution), is injected into the anterior chamber of the eye before and/or during sonication, resulting in a substantial reduction in temperature rise, and peak temperature, thus avoiding thermal injury. The application of HIUE in an aqueous medium, however, has been proven to generate acoustic cavitation (Ref 1).

The alternating pressure induced by HIUE on the dissolved bubbles of gas in the irrigating solution can lead to acoustic cavitation, an adiabatic rapid compression of gas bubbles that converts the potential energy of the bubbles into chemical, electromagnetic, thermal, and mechanical energies, in the form of reactive oxygen species (ROS), sonoluminescence, a rise in temperature, and localized extreme pressures, respectively (Ref 2). The main anticipated chemical effects of acoustic cavitation are mediated through ROS.

Phacoemulsification is the preferred and most commonly used surgical treatment for cataract. About 1.5 million cataract removal operations are performed annually in the U.S., with a similar figure in Europe and the rest of the world. The main advantage of this technique is the small incision (3.0 mm) required for lens removal and implantation of a foldable intraocular lens. However, damage to the endothelial monolayer of the cornea resulting from phacoemulsification in cataract removal surgery has been reported. Such damage, which might include destruction of non-regenerative endothelial cells, is of major significance. The transparency of the cornea is maintained by the corneal endothelium, which serves as a physical barrier to the movement of fluid and as an active pump across the cornea. Numerous studies have recorded a significant decrease in endothelial cell density and increase in corneal thickness as a result of phacoemulsification.¹ These injuries were attributed mainly to the mechanical effects of ultrasound and of the lens fragments in the anterior chamber. Since human corneal endothelial cells lack the ability to regenerate, severing the corneal endothelium may lead to lasting corneal damage and permanent edema in the form of irreversible bullous keratopathy.

ROS participate in multiple biological reactions, such as mutation, carcinogenesis, aging, cataract formation, inflammation, and various diseases,³ which can lead to immediate cell killing, necrosis, apoptosis, or other forms of cell damage. A previous study by our group demonstrated the generation of ROS by acoustic cavitation under in vitro conditions simulating cataract surgery. More recently we demonstrated that endothelial cell loss after phacoemulsification in an in viva rabbit model could be reduced by 70% by the addition of ascorbic acid to the irrigating solution, suggesting that the mechanism of damage induced by phacoemulsification is at least partly chemical.

The addition of antioxidants may significantly, dose dependently alter the biocompatible nature of the irrigating solution by lowering of the pH of the irrigating solution to acidic level as demonstrated in Table (1).

TABLE (1)

| | pH | Temp C.° |
|---|---|---|
| BSS (Alcon, USA) + | | |
| Ascorbic Acid (Sigma, Israel Ltd.) | | |
| BSS | 6.91 | 21.8 |
| BSS + AA 10⁻¹M | 3.92 | 21.5 |
| BSS + AA 10⁻²M | 5.23 | 22.2 |
| BSS + AA 10⁻³M | 6.25 | 22.2 |
| BSS + AA 10⁻⁴M | 6.77 | 21.2 |
| BSS (Alcon, USA) + | | |
| Oxidized Glutathione | | |
| (GSSG)(Sigma, Israel Ltd.) | | |
| BSS + GSSG 10⁻²M | 4.84 | 19.7 |
| BSS + GSSG 10⁻³M | 6.07 | 20.1 |
| BSS + GSSG 10⁻⁴M | 6.73 | 19.8 |
| BSS (Alcon, USA) + Reduced | | |
| Glutathione (GSH)(Sigma, | | |
| Israel Ltd.) | | |
| BSS + GSH 10⁻²M | 5.20 | 19.9 |
| BSS + GSH 10⁻³M | 6.28 | 20.2 |
| BSS + GSH 10⁻⁴M | 6.80 | 20.5 |

The use of (scavengers) antioxidants in oral administration, such as Vitamins A, C, D, E and others, was proposed prior to or after exposure to sonication in order to reduce the possible effects of free radicals. The limitation of the oral application of scavengers lies in the need for high concentrations of these substances to avoid the effects of free radicals. This method does not apply to phacoemulsification as the anterior chamber fluid is instantly flushed at the start of the irrigation of the anterior chamber eliminating all natural occurring antioxidants in the anterior chamber. Although the addition of sufficient concentration of antioxidants to the sonicated medium does not inhibit cavitation it can effectively reduce damage of radicals to the corneal endothelium as was shown in a rabbit eye model.

In order to demonstrate the protective effects of antioxidants in reducing corneal endothelial cell loss secondary to phacoamulsification even in the absence of a gas selected from the group consisting of carbon dioxide, nitrogen, or mixtures thereof, the effects of antioxidants in a biocompatible buffered solution was evaluated. Seventeen eyes of seventeen New-Zealand white female rabbits (suppl), weighing 3 to 4 kilograms each, were exposed to high energy ultrasound energy by the activation of a phacoemulsification probe in the anterior chamber, Treatment and handling of the rabbits was carried out in accordance with the ARVO Statement for the Use of Animals in Ophthalmic and Vision Research. Prior to surgery, the rabbits were anesthetized by intramuscular injection of ketamine hydrochloride (Ketalar) 35 mg/kg and xylazine (Rompun) 7 mg/kg, and the right eye was then examined by specular microscopy (Tomey EM-1000 Specular Microscope, Tomey, Japan), obtaining three corneal endothelial photographs for analysis, all from within the central 3 mm of cornea. The right eye was cleansed with topical iodine (Betadine), draped and a lid speculum was inserted. Next, a clear corneal incision was made in the superotemp oral corneal quadrant with a disposable 3.2 mm keratome blade (3.2 mm Surgistar symmetry knife, Surgistar, Knoxville, Tenn.). Using 70% power, and 25 ml/min of irrigation, a 20 g phacoemulsification probe (Series Ten Thousand Phacoemulsification System, Alcon Surgical, Fort Worth, Tex.) was introduced via the corneal incision into the anterior chamber, taking care to avoid touching all ocular structures including the lens and cornea, and activated in the center of the anterior chamber. Each eye was continuously irrigated, with the power turned alternately on and off every 10 seconds to avoid overheating, until the required 5 minutes of net time (10 minutes in all) was completed.

The rabbits were randomized to recieve either balanced salt solution (BSS, Alcon Surgical, USA), or BSS with 0.001M of sterile ascorbic acid (Cereon, Teva Pharmaceuticals, Petach-Tikva, Israel). Ascorbic acid was introduced into the BSS bottle immediately prior to phacoemulsification. The two groups were similar with regard to all other respects of surgical and postoperative treatment. Each animal was number encoded prior to treatment, and surgery, postoperative and histological examinations and analysis were all performed with blinding as to the treatment grouping.

On completion of phacoemulsification, the incision was sealed by injection of BSS solution (for both groups) into the incision margins, followed by a subconjunctival injection of betamethasone acetate and betamethasone phosphate (Celestone Chronodase, Scering-Plough, Kenilworth N.J.) and gentamicin (Gentamicin-IKA, Teva Pharmaceuticals, Israel) was given. No additional postoperative medications were given.

One week later the rabbits were again anesthetized as described above, the operated eye was again examined by specular microscopy, and the rabbits were then sacrificed by an overdose of phenobarbital. The right eye of each rabbit was enucleated and preserved in formaldehyde, and corneal sections were stained with hematoxylin and eosin and examined by an experienced ocular pathologist who was blinded to the treatment received by each rabbit, as mentioned previously.

The specular microscopy results were analysed using the EM-1100 software application for endothelial cell analysis (EM-1100 endothelial cell analysis software, Tomey, Japan). The results of the 3 preoperative and 3 postoperative endothelial cell counts were averaged separately and the reduction in cell count 1 week after surgery calculated by subtraction. The non-parametric Mann-Whitney test was used to examine possible differences in preoperative cell counts between the two groups, as well as differences in postoperative cell count reduction. Cell counts are presented as mean±SEM.

Preoperative and postoperative endothelial cell counts are shown in Table (2), in random and non-chronological order within each group.

It should be noted that It was previously found that the normal inter-examination variation between endothelial cell counts to be about ±5%, and postoperative results showing minor increases in endothelial cell counts are probably due to this variation.

TABLE (2)
Specular microscopy results.

| | | | | % change |
|---|---|---|---|---|
| Eye | Irrigating | Pre-operative | Post-operative | in endothelial |
| no. | Solution | cell count/mm³ | cell count/mm³ | cell density |
| 1 | BSS* | 3011 | 2381 | −20.9 |
| 2 | BSS | 3294 | 3027 | −8.1 |
| 3 | BSS | 3000 | 2390 | −20.3 |
| 4 | BSS | 3631 | 3112 | −14.3 |
| 5 | BSS | 3058 | 2205 | −27.9 |
| 6 | BSS | 3199 | 2908 | −9.1 |
| 7 | BSS | 3123 | 2914 | −6.7 |
| 8 | BSS | 3135 | 2883 | −8.0 |
| 9 | BSS + AA† | 3058 | 3195 | +4.7 |
| 10 | BSS + AA† | 2756 | 2835 | +2.8 |
| 11 | BSS + AA† | 3021 | 3170 | +4.9 |
| 12 | BSS + AA† | 2885 | 2655 | −7.9 |
| 13 | BSS + AA† | 3493 | 3363 | −3.7 |
| 14 | BSS + AA† | 3525 | 3154 | −10.5 |
| 15 | BSS + AA† | 2968 | 2765 | −6.8 |
| 16 | BSS + AA† | 3082 | 2854 | −7.4 |
| 17 | BSS + AA† | 3137 | 2825 | −9.9 |

*Balanced Salt Solution
†BSS with 10⁻³M ascorbic acid
The statistical test revealed no differences between the two groups in preoperative

cell counts at the 5% significance level (Mann-Whitney test P=0.37).

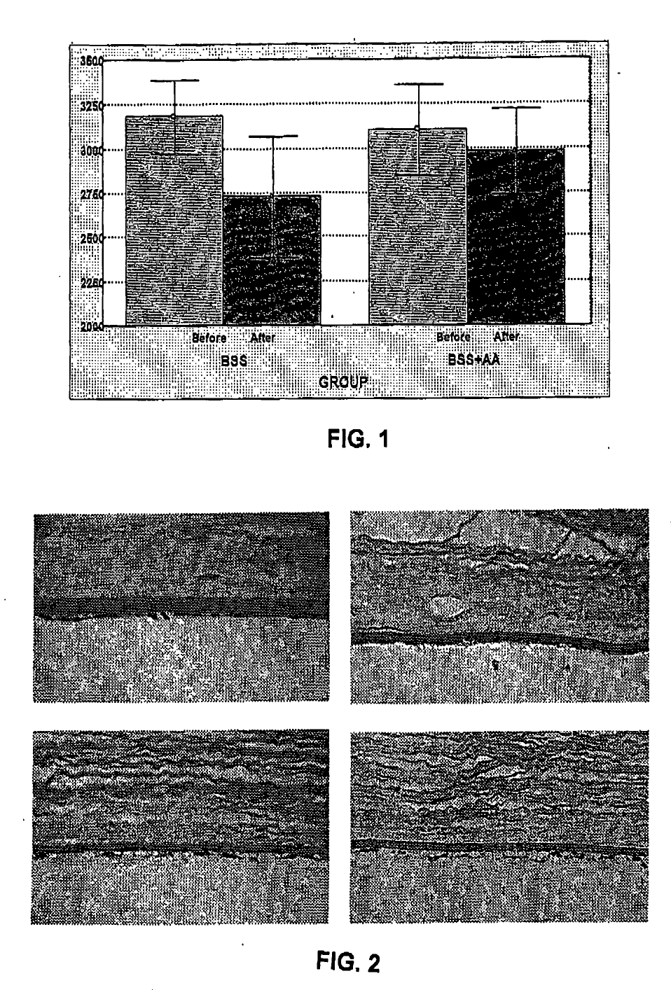

The reduction in endothelial cell count (cells/mm²) one week after surgery was 453.9±233.3 (SEM) in the group that received BSS and 123.2±196.4 (SEM) in the group treated with BSS plus ascorbic acid (FIG. 1).

Referring to FIG. 1 there is seen a graphical representation of preoperative and postoperative endothelial cell counts groups treated with BSS and with BSS with ascorbic acid (BSS+AA) group.

The postoperative reduction in cell count differed significantly between the two groups, (Mann-Whitney test P=0.011). Thus the cell count was significantly higher in the group treated with BSS+10⁻³ M ascorbic acid than in the group treated with BSS alone. The mean reduction in cells count in the BSS group was more than 3.5-fold greater than in the group treated with BSS+AA.

FIG. 2 presents 4 histological slides prepared from all corneas from rabbits in the two treatment groups which showed marked differences In endothelial cell morphology. Endothelial cells from the central corneas in the BSS group contained more and much larger vacuoles than those from the group treated with BSS+AA (FIG. 2). For each group, similar findings were obtained in all ten central corneal sections examined.

Referring to FIG. 2 there are shown postoperative central corneal sections taken from the two groups (magnification times 400). The top two slides show post-operative corneas from the group treated with BSS+AA, while the two bottom slides show corneas from the group treated with BSS. The two latter slides show a marked decrease in endothelial cell density as well as marked endothelial cell vacuolization.

The addition of buffered biocompatible sterile ascorbic acid to the irrigating solution resulted in a pH of 6.91 and has significantly reduced the amount of endothelial cell loss during phacoemulsification by about 70%. It is due to the free radical scavenging properties of ascorbic acid.

In a separate study, it was demonstrated that the protective effects of antioxidants were diminished and became virtually destructive by the addition of high concentrations of antioxidants to the irrigating solution. It is demonstrated in the following summary of the effects of phacoemulsification applying irrigating solution containing GSSG In a concentration of 10⁻² M with a pH of 4.84, applied in a model group of four rabbit eyes. In this group a 24.5% cell loss was observed following phacoemulsification, emphasizing the damaging effect of the non-buffered antioxidant solutions.

In another study applying lower concentrations of GSSG it was demonstrated that the effects of phacoemulsification applying irrigating solution containing GSSG in a concentration of 10⁻³ M with a pH of 6.05, applied in a model group of eight rabbit eyes, only a 7.7% cell loss was observed. This proves the direct correlation between the acidity of the solution and the damaging effect of the non-buffered acidic antioxidant solutions. The addition of bicarbonate as a buffer to the irrigating solution maintaining a pH of 6.85 reduced endothelial cell loss to 2.4%.

The addition of anti-oxidants to the irrigating solution lowers the pH of the irrigating solution to acidic level causing irreversible damage to the corneal endothelium through direct chemical damage. As seen in table (1), solutions containing M at a concentration of 10⁻⁴ M has a pH of 6.77 inflicting chemical damage to the corneal endothelium substantially reducing its advantageous antioxidative effect.

It is for this reason that the present invention now provides a method for using a biocompatible injectable solution in high intensity ultrasound energy assisted surgery wherein said solution comprises antioxidants selected from the group consisting of ascorbic acid, glutathione and mixtures thereof in a buffered biocompatible solution for the reduction and limitation of reactive oxygen species.

The present invention provides a method for using a biocompatible injectable sterile aqueous solution in phacoemulsification wherein said solution comprises antioxidants selected from the group consisting of ascorbic acid, glutathione and mixtures thereof in a buffered biocompatible solution for the reduction and limitation of reactive oxygen species

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

### EXAMPLE 1

As the addition of antioxidants alter the pH of the irrigating solution a biocompatible buffer, such as a bicarbonates is applied to the solution to maintain the pH in its physiological range as demonstrated in table (3). Sodium bicarbonate was added in an amount sufficient to achieve the adequate biocompatible pH as shown in table (3).

TABLE 3

| BSS (Alcon USA, Ltd) + Vit. C (Teva, | | |
|---|---|---|
| Israel, Ltd.) (Buffered Ascorbic | | |
| Acid) | pH | Temp C.° |
| BBS + Vit. C 10⁻²M | 6.84 | 22.1 |
| BSS + Vit. C 10⁻³M | 6.91 | 22.2 |
| BSS + Vit. C 10⁻⁴M | 6.92 | 22.1 |

It will thus be noted that by adding a buffer to create a buffered solution it is possible to form a safe and effective biocompatable injectable aqueous solution which will not cause damage to the corneal endothelium and which therefore enables the use of such antioxidants as ascorbic acid, glutathione and mixtures thereof in high intensity ultrasound energy assisted surgery and especially for use in phacoemulsification.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### REFERENCES

1. Topaz M, Motiei M, Gedanken A, Meyerstein D, Meyerstein N. EPR analysis of radicals generated in ultrasound-assisted lipoplasty simulated environment. Ultrasound Med Biol. 2001; 27:851-859
1. Topaz M. Possible long term complications in ultrasound-assisted lipoplasty induced by sonoluminescence, sonochemistry, and thermal effect. Long-term possible hazardous effects of ultrasonically assisted lipoplasty. Plast Reconstr Surg. 1998; 102:280-281.
1. Topaz M, Motel M, Assia E, Meyerstein D, Mayerstein N, Gedanken A. Acoustic cavitation in phacoemulsification: chemical effects, modes of action and cavitation index. Ultrasound Med Biol. 2002; 28(6): 775-84.

1. A biocompatible, injectable aqueous solution for use in high-intensity-ultrasound-energy-assisted surgery comprising antioxidants selected from the group consisting of ascorbic acid, glutathione and mixtures thereof in a buffered biocompatible solution for the reduction and limitation of reactive oxygen species.

2. A biocompatible, injectable aqueous irrigation solution for use in phacoemulsification comprising ascorbic acid in a buffered biocompatible solution for the reduction and limitation of reactive oxygen species.

3. A method for using a biocompatible injectable aqueous solution in high intensity ultrasound energy assisted surgery wherein said solution comprises antioxidants selected from the group consisting of ascorbic acid, glutathione and mixtures thereof in a buffered biocompatible solution for the reduction and limitation of reactive oxygen species.

4. A method for using a biocompatible injectable aqueous solution in phacoemulsification wherein said solution comprises antioxidants selected from the group consisting of ascorbic acid, glutathione and mixtures thereof in a buffered biocompatible solution for the reduction and limitation of reactive oxygen species.
